SEQUENCE LISTING (10 sequences)

SEQ ID NO: 1 - DNA, 26 bp, Artificial
  Forward PCR primer
gcagcagtgggaactccaaggagaca

SEQ ID NO: 2 - DNA, 20 bp, Artificial
  Reverse PCR primer
gcatgagatgttgctcgcag

SEQ ID NO: 3 - DNA, 21 bp, Artificial
  Peanut DNA specific probe
cgagagggcgaacctgaggcc

SEQ ID NO: 4 - DNA, 21 bp, Artificial
  Competitor DNA specific probe
ccggagtccaagcgggagagc

SEQ ID NO: 5 - DNA, 76 bp, Artificial
  Synthesis oligo #1
gcagcagtgggaactccaaggagacagaagatgccagagccagctgcaagggcggccggagtccaagcgggagagc

SEQ ID NO: 6 - DNA, 52 bp, Artificial
  Synthesis oligo #2
gcatgagatgttgctcgcaggcgcctagtgtgctctcccgcttggactccgg

SEQ ID NO: 7 - DNA, 86 bp, Artificial
  Probe
gcagcagtgggaactccaaggagacagaagatgccagagccagctcgagagggcgaacctgaggccctgcgagcaacatctcatgc

SEQ ID NO: 8 - DNA, 21 bp, Artificial
  Primer
ccggagtccaagcgggagagc

SEQ ID NO: 9 - DNA, 107 bp, Artificial
  Probe
gcagcagtgggaactccaaggagacagaagatgccagagccagctcgagagggcgccggagtccaagcgggagagcaacctgaggccctgcgagcaacatctcatgc

SEQ ID NO: 10 - DNA, 107 bp, Artificial
  Probe
gcagcagtgggaactccaaggagacagaagatgccagagccagctgcaagggcggccggagtccaagcgggagagcacactaggcgcctgcgagcaacatctcatgc

(19) United States
(12) Patent Application Publication
(10) Pub. No.: US 2011/0159495 A1
(43) Pub. Date: Jun. 30, 2011

(54) METHOD FOR THE QUANTITATIVE DETECTION OF AN ORGANIC SUBSTANCE IN A SAMPLE

(76) Inventor: Thomas Holzhauser, Langen (DE)

(21) Appl. No.: 13/061,907

(22) PCT Filed: Sep. 7, 2009
(86) PCT No.: PCT/EP09/61584

§ 371 Date: Mar. 2, 2011

(30) Foreign Application Priority Data
Sep. 5, 2008 (EP) 08015740.7
Dec. 2, 2008 (EP) 08020918.2

(51) Int. Cl. C12Q 1/68

(52) U.S. Cl. 435/6.11; 435/6.12

(57) ABSTRACT

A method for the quantitative detection of an organic substance containing a nucleic acid to be detected in a sample based on a given threshold value for that organic substance in the sample, the use of this method, as well as a kit for practicing this method.

### FIELD OF THE INVENTION

The invention relates to a method for the quantitative detection of an organic substance in a sample, which method is based on isolated nucleic acid from said sample. The invention relates in particular to a method and a kit of parts for the quantitative detection of an organic substance of plant or animal origin in a sample.

### BACKGROUND OF THE INVENTION

There is presently no causative therapy available for individuals that are allergic to certain foods. Therefore, the only effective measure to avoid a potentially life-threatening allergic reaction of an individual to a certain food is to strictly avoid the consumption of the allergenic food itself. This requires full transparency of food ingredients and food constituents, respectively. Although the labeling of certain allergenic food ingredients is governed by law in many Western and Asian countries, not all known allergenic food ingredients need to be labeled. In addition, so-called allergen cross-contamination of non-allergenic foods with allergenic foods may occur during production processes of food. Consequently, to verify legal requirements as well as to control allergen cross-contamination, sensitive, specific, and quantitative methods are needed to screen for the presence of and to determine the amount of allergenic food constituents.

Detection and quantitation of potentially allergenic food ingredients is mainly based on the detection of a specific protein using enzyme-linked immunosorbent assays (ELISA). However, such assays have been demonstrated to provide semi-quantitative results only (Poms et al., Food additives and contaminants 2005, 22: 104-112).

Nucleic acid-based methods and the polymerase chain reaction (PCR) have further been used for detection of allergenic food constituents (Holzhauser et al., Eur. Food Res. Technol. 2000, 211: 360-365). Furthermore, the gel-based detection of PCR-products for the purpose of quantification is disadvantageous because the gels are not very reproducible, require the use of potentially mutagenic fluorescent dyes, and the handling of PCR amplicons in a laboratory is a potential source for cross-contaminations of other PCR samples. Real-time PCR requires no post-PCR manipulation of the amplicons and is therefore preferably used (Hird et al., Eur. Food Res. Technol. 2003, 217: 265-268; Stephan and Vieths, J. Agric. Food Chem. 2004, 52: 3754-3760). While PCR is an exquisitely sensitive method for detection of minute amounts of target DNA, the exponential nature of DNA amplification, however, is prone to burden the experimental data with significant standard error. This is due to the tube-to-tube inherent variations in amplification efficiency and because PCR-based methods generally require an extraction of the nucleic acids from the sample, which is most variable in the case of complex multi-compound sample matrixes, such as food and food products. The yields of extracted DNA are dependent of the sample matrix and also inhibitory components may become co-extracted and affect the efficiency of DNA amplification within each cycle of amplification. Due to the number of dependent cyclic reactions, the error from sample preparation will also be amplified exponentially so that this method is rarely used, if at all, to determine the amount of an organic substance in a sample such as an allergenic food constituent. Classical PCR is not suitable for quantification, since it is an end-point method, wherein the amplification is done until saturation of each PCR-reaction, which does not allow quantitative differentiation. Real-time PCR needs to be extremely well controlled in order to obtain accurate quantification. In practical application, it is therefore not sufficiently accurate and in effect an extremely poor method when it comes to the quantification of an organic substance in a sample.

Thus, it has been proposed to add a known amount of an internal standard DNA or a reporter gene which is co-amplified which a quantitation of the target DNA is required (Zacher et al., Nucleic Acids Research 1993, 21: 2017-2018). This method however requires a gel analysis of the amplified products and post PCR manipulation of the amplicons and can therefore not be used in a real-time PCR. It has been proposed to include an internal standard in the PCR amplification.

While PCR is an exquisitely sensitive method for detection of minute amounts of target DNA, the exponential nature of DNA amplification, however, is prone to burden the experimental data with significant standard error due to the tube-to-tube inherent variations in amplification efficiency. Therefore the reliable quantitation of DNA or RNA templates by PCR necessitates introduction of an internal standard. Typically, in a series of replicates of analyzed sample the defined range of such standards is co-amplified together with constant unknown amount of wild-type template or alternatively both templates are subjected to endpoint dilution. Obviously, each of the methods is based on multiple PCR reactions which are to be performed simultaneously with each of the analyzed samples. Such procedure, nevertheless, may become cumbersome when large numbers of samples are to be analyzed (Wei-Liu et al., Nucl. Acids Res. 2009, 37, 1: e4).

Quantitative real-time PCR is known for cell cultures of human carcinoma (Gibson et al., Genome Research 1996, 6, 10, 995-1001). However, this method requires a competitor with a reporter fluorescent dye as an internal control. In order to calibrate the internal control against the target sequence, a dilution series of the internal control with equal amounts of the target sequence is prepared and analysed, and a calibration curve is derived from the measurements obtained. In the example presented, the dilution series contains about 15 solutions. The calibration is therefore extremely cumbersome, error-prone and time consuming. Similar methods are known for the determination of genetically modified soybeans (DE 199 06 169 A1) and human herpesvirus (Secchiero et al., J. Clin. Microbiol. 1995, 33, 8, 2124-2130). The example on genetically modified soybeans further requires the availability of external reference material with known amounts of the GMO material under investigation.

All these methods have in common that a separate calibration using copies of the detection target is required. Furthermore, the methods are only described for the application in a single matrix without demonstrating transferability to other matrices, or complex multi-compound matrices, and are thus not generally applicable.

The prior art therefore poses a problem.

### SHORT DESCRIPTION OF THE INVENTION

The problem is solved by a method for the quantitative detection of an organic substance in a sample based on a given threshold value for that organic substance in the sample through an amplification of a nucleic acid to be detected that stems from said organic substance, comprising the steps of (a) providing a given number of a second nucleic acid as a competitor (competitor nucleic acid) in an amplification of the nucleic acid to be detected, and adding the competitor nucleic acid to the sample, wherein the given number of the competitor nucleic acid results (eg through co-extraction and co-amplification in comparison to the extraction, and amplification of the nucleic acid of the organic substance) in a signal that allows for deducing the amount of the organic substance in the sample relative to the given threshold value; (b) extracting the nucleic acid to be detected and the competitor nucleic acid from the sample; (c) providing a first oligonucleotide and a second oligonucleotide for priming an amplification of the nucleic acid to be detected and of the competitor nucleic acid; (d) providing a first detectable probe for hybridizing with the nucleic acid to be detected to generate a first detectable signal; and a second detectable probe for hybridizing with the competitor nucleic acid to generate a second detectable signal; (e) performing an amplification of the nucleic acid to be detected and simultaneously of the competitor nucleic acid; and (f) measuring the signal from the first detectable probe and the signal from the second detectable probe and deducing therefrom the amount of the organic substance in the sample relative to the given (calibrated) threshold value. The amplification is performed through a real-time polymerase chain reaction.

In one preferred embodiment of the invention, the deduction of the amount of the organic substance in the sample is performed by determining the C_{T} value of the signal from the first detectable probe and of the signal from the second detectable probe representing the threshold value, and by comparison of the C_{T} value from the first detectable probe with the C_{T} value from the second detectable probe to deduce the amount of the organic substance in the sample relative to the threshold value by application of the following steps: (a) in case the C_{T} value from the first detectable probe is substantially identical to the C_{T} value from the second detectable probe, it is concluded that the amount of the organic substance in the sample is identical to the threshold value; (b) in case the C_{T} value from the first detectable probe is below the C_{T} value from the second detectable probe, it is concluded that the amount of the organic substance in the sample is higher than the threshold value; and (c) in case the C_{T} value from the first detectable probe is above the C_{T} value from the second detectable probe, it is concluded that the amount of the organic substance in the sample is lower than the threshold value.

If the PCR efficiency for both the organic substance and the competitor is identical and nearly 100%, a difference in C_{T} value from the first detectable probe to the C_{T} value from the second detectable probe by 3.322 cycles will result in a relative difference of tenfold compared to the calibrated amount.

In a further advantageous embodiment of the invention, the nucleic acid to be detected and the competitor nucleic acid both comprise a first sequence portion for hybridization with the first oligonucleotide, and a second sequence portion for hybridization with the second oligonucleotide, and the first sequence portion of the nucleic acid to be detected or of the competitor nucleic acid contains a sequence that is reverse complementary to the sequence of the first oligonucleotide, or the second sequence portion of the nucleic acid to be detected or of the competitor nucleic acid contains a sequence that is reverse complementary to the sequence of the second oligonucleotide.

In further advantageous embodiments, the first sequence portion of the nucleic acid to be detected and the first sequence portion of the competitor nucleic acid are identical. Alternatively, the second sequence portion of the nucleic acid to be detected and the second sequence portion of the competitor nucleic acid are identical. Alternatively, the amplification product of the nucleic acid to be detected and of the competitor nucleic acid are identical in length. Alternatively, the amplification product of the nucleic acid to be detected and the competitor nucleic acid differ in sequence in at least 1 nucleotide, preferably in 1 to 20 nucleotides. Alternatively, the nucleic acid to be detected and the competitor nucleic acid differ in sequence only at a sequence portion where the detectable probe hybridizes.

In a further embodiment of the invention, the first detectable probe and the second detectable probe are tagged with a detectable label, and the detectable label of the first detectable probe is distinguishable form the detectable label of the second detectable probe.

In yet another embodiment of the invention, the quantitative detection is an absolute quantitative detection.

Also part of the invention is a packaging unit or kit for the quantitative detection of an organic substance in a sample wherein a target nucleic acid, which presence correlates with the amount of organic substance, is isolated from said sample and quantified by real-time PCR to determine whether the organic substance was comprised at, above, or below a set threshold value in said sample, comprising (i) at least one vessel for calibration of the detection system comprising defined amounts of said target nucleic acid and a competitor nucleic acid in such a ratio that their C_{T}-values can be laid over each other when quantified in a standardised real-time PCR, (ii) at least one vessel comprising a defined amount of competitor nucleic acid to be added to a defined amount of sample to be extracted or at least one extraction vessel comprising a defined amount of competitor into which a defined addition of the sample to the defined amount of competitor nucleic acid is done prior to nucleic acid extraction, so that the defined amount of competitor nucleic acid serves as an internal standard for nucleic acid extraction and amplification from said sample and is chosen such that the amount of competitor sets an internal reference to the quantitative threshold of the organic substance when a defined amount of sample is analysed by nucleic acid extraction and real-time PCR with respect to the presence of target and competitor nucleic acids, and (iii) optionally one or more amplification vessels comprising first and second primers, reagents and enyzmes for nucleic acid extraction and for performing a standardized real-time PCR.

In a preferred embodiment of the invention, the packaging unit or kit further comprises one or more vessels for holding said sample and subsequent nucleic acid extraction, wherein each vessel comprises a defined amount of competitor nucleic acid that sets an internal threshold when the target and competitor nucleic acids are analysed by real-time PCR.

In a further preferred embodiment of the invention, the amplification vessels further comprise first and second detectable probes which hybridise with the target nucleic acid and the competitor nucleic acid respectively, and which are labelled with first and second detectable labels respectively chosen, for example, from FAM, HEX, Cy3 or Cy5, such that the detectable label of the first probe is distinguishable from the detectable label of the second probe.

In a further preferred embodiment of the invention, the packaging unit or kit is characterised in that the vessels are prepared such that the defined amount of competitor nucleic acid is incorporated i) in a liquid solution phase, or ii) as a material reversibly bound to an enclosed solid phase to be added, or iii) in a glassy layer of trehalose bound to the vessel walls.

In a further preferred embodiment of the invention, the packaging unit or kit is characterised in that the vessels are prepared such that the defined amounts of competitor and target nucleic acid are incorporated i) in a liquid solution phase, or ii) as a material reversibly bound to an enclosed solid phase to be added, or iii) in a glassy layer of trehalose bound to the vessel walls of the calibration vessel.

In one preferred embodiment of the invention, the reaction vessels in the packaging unit or kit are appropriately sized micro-reaction tubes or wells of a microtiter plate.

Furthermore, the use of the method according to the invention and the use of the packaging unit or kit according to the invention also forms part of the present invention.

In one especially preferred embodiment of the invention, the kit or packaging unit is adapted for the simultaneous quantitative detection of two or more organic substances in a sample, wherein respective target nucleic acids, the presence of which correlates with the amount of organic substances, are isolated from said sample and quantified by real-time PCR to determine whether the organic substances were comprised at, above, or below a set threshold value in said sample, comprising (i) at least one vessel for calibration of the detection system comprising defined amounts of said target nucleic acids and respective competitor nucleic acids in such a ratio that their respective C_{T}-values can be laid over each other when quantified in a standardised real-time PCR, (ii) at least one vessel comprising defined amounts of competitor nucleic acids to be added to a defined amount of sample to be extracted, or at least one extraction vessel comprising defined amounts of competitors into which a defined addition of the sample to the defined amount of competitor nucleic acids is done prior to nucleic acid extraction, so that the defined amounts of competitor nucleic acids serve as an internal standards for nucleic acid extraction an amplification from said sample and are chosen such that the amounts of competitor set internal references to the quantitative thresholds of the organic substances when a defined amount of sample is analysed by nucleic acid extraction and real-time PCR with respect to the presence of target and competitor nucleic acids, and optionally (iii) one or more vessels comprising primers, reagents and enyzmes for nucleic acid extraction and for performing a standardized real-time PCR, wherein the probes are respectively labelled with detectable labels comprising probes chosen such that all the detectable labels are distinguishable, so as to allow relative quantification of each target nucleic acid compared to its respective competitor nucleic acid.

The method and kit according to the invention allow the quantification of an organic substance of plant or animal origin in composed matrices such as food, feed, body fluid, and stool samples, and in particular, an easy quantitative detection of allergenic food component in a sample wherein method and means are based on the amplification and detection of nucleic acid stemming from the species causing the allergic reaction.

The method is particularly useful for detecting and quantifying allergens from certain plants or animals in food products because there is no necessity of external calibration. This is because the method is based on a given threshold value for that organic substance in the sample, which represents a certain amount or concentration of the organic substance in the sample. The method makes use of an amplification reaction of a first nucleic acid, the nucleic acid to be detected in the sample that stems from said organic substance. The method comprises the following steps:

Firstly, a given number of a second nucleic acid molecule is provided as a competitor for an amplification of the nucleic acid to be detected. The provided competitor molecule is added to the sample prior to extracting the nucleic acid to be detected from the sample. In an amplification reaction, this second nucleic acid will serve as a second template besides the nucleic acid to be detected. Thereby, the competitor nucleic acid serves as an internal standard for normalising the extraction and amplification reaction. In an amplification reaction, the given number of the competitor nucleic acid results in a signal that allows for deducing the amount of the organic substance in the sample relative to the given analytical threshold value.

Secondly, a first oligonucleotide molecule and a second oligonucleotide molecule, both for priming an amplification of the nucleic acid to be detected, is provided. The first and the second oligonucleotide should be present in excess over the template molecules, so that the amplification reaction is not impeded due to a lack of oligonucleotides that serve as primers for the amplification reaction. The first and the second oligonucleotide have a nucleotide sequence that allows them to hybridize to the nucleic acid to be detected and to the competitor nucleic acid to prime amplification reactions. It is preferred that the hybridization sites of the first and the second oligonucleotide on the template molecules, i.e. the nucleic acid to be detected and the competitor nucleic acid, are identical for the first oligonucleotide and/or the second oligonucleotide.

Thirdly, the nucleic acid to be detected is extracted from the sample using a suitable extraction method. Since the competitor nucleic acid was previously added to the sample, the competitor nucleic acid is also extracted from the sample with the same or similar efficiency as the nucleic acid to be detected, since the competitor nucleic acid is subject to the same interactions with the sample's matrix material as the nucleic acid to be detected. The amount of competitor nucleic acid necessary for generating an appropriate signal in an amplification reaction needs to be determined prior to the first quantification of the organic substance to be detected. Once determined, the amount of competitor nucleic acid that is added is kept constant in relation to the amount of sample from which the nucleic acid is to be extracted.

Any known extraction method for extracting and purifying nucleic acids from matrix material, such as silica-based nucleic acid extraction or classic CTAB and/or phenol and chloroform extraction, can be applied. Once selected, the applied method for nucleic acid extraction and purification and the down-stream PCR amplification reaction will need to be performed in a reproducible way with the predetermined amount of competitor nucleic acid present throughout nucleic acid extraction to detection. The term matrix material is meant to refer to the specimen in which the nucleic acid to be analyzed is present in the sample.

Fourthly, a first detectable probe for hybridizing with the nucleic acid to be detected is provided, which can generate a first detectable sequence-specific signal. Also, a second detectable probe for hybridizing with the competitor nucleic acid is provided to generate a second detectable sequence-specific signal. By hybridizing with the amplification products, the probes emit a detectable signal. In order to differentiate which signal stems from the amplification of which nucleic acid molecule, the first detectable probe is labelled differently than the second detectable probe. The relative difference between the position of the specific signal of the nucleic acid to be detected and of the competitor nucleic acid is a means to quantify the organic substance in a ratio to the analytical threshold set up initially.

When the amplification is performed using real-time polymerase chain reaction, the amplification of nucleic acids results in a certain position of a sequence-specific signal of amplification of the nucleic acid to be detected in a graph showing signal over time (or number of amplification cycles). The position of sequence specific amplification for both types of amplifiable nucleic acid, target and competitor, is preferably determined at an identical or predefined magnitude of signal. The position of sequence specific amplification at this predefined magnitude of signal is called “threshold cycle”.

Fifthly, an amplification reaction is performed, in which both the nucleic acid to be detected and the competitor nucleic acid are simultaneously amplified, using the same oligonucleotides as primers. The amplification is preferably an enzymatic amplification. In case the enzyme based amplification reaction is a polymerase based amplification reaction, in particular a polymerase chain reaction (PCR), it is preferred that the enzyme is a heat-stable polymerase. However, it is also possible to apply other enzymatic amplification reactions know to a person of skill in the art, including ligase-mediated amplifications (e.g. ligase-chain reaction) or amplifications based on transcription (e.g. NASBA®, 3SR®, TMA®). As an enzyme, a polymerase, such a Taq or Pfu or a ligase can be used, as appropriate for the amplification reaction performed.

Sixthly, the signal stemming from a first detectable probe and the signal stemming form the second detectable probe are detected for determining the quantitative amount of the organic substance under investigation, based on its nucleic acid to be detected. There from, the amount of the organic substance in the sample relative to the given threshold value is deduced. Preferably, in case a real-time PCR is used, the position of the signal originating from the nucleic acid to be detected in relation to the position of the signal originating from the competitor nucleic acid is determined.

In short, the invention provides for a means for quantifying an organic substance without quantifying its nucleic acid, e.g. from a food component, by providing an internal standard that is carried along during both the extraction and the analysis phase of the sample. When the method is performed using real-time PCR, the difference of the position (threshold cycle) of a sequence specific signal originating from a nucleic acid to be detected in relation to a competitor nucleic acid is detected by providing an internal standard that is carried along during both the extraction and the analysis phase of the sample. The internal standard has an amplification efficiency that is identical or nearly identical to the amplification efficiency of the nucleic acid to be detected. The term “amplification efficiency” refers to the binding efficiency of the nucleotides used to prime an amplification reaction and the efficiency of the amplification reaction itself, e.g. the factor that the number of molecules of a particular template species is amplified by in each round of the amplification reaction.

With the method of the invention, the disadvantage of potentially non-reproducible antibody preparations and protein standards that are used in protein-based detection methods such as enzyme immunoassays is encountered by the use of a DNA-based methodology, such as polymerase chain reaction (PCR), of which the complete chemistry including recombinant polymerase enzyme is of a reproducible and therefore consistent quality.

The nucleic acid is preferably DNA, but can also be RNA, such as mRNA that is subject to a reverse transcription reaction prior to the amplification reaction,

Ideally, the template nucleic acid, be it the nucleic acid to be detected or the competitor nucleic acid, does not exceed 200 base pairs (bp) in length. Preferred is a length of between 50 bp to 180 bp. The first and/or second oligonucleotide for priming the reaction should have a length of between 12 bp to 30 bp in length. The first and/or second detectable probe should not exceed 40 bp in length.

In a preferred embodiment of the method according to the invention, the nucleic acid to be detected and the competitor nucleic acid both comprise a first sequence portion for hybridisation with the first oligonucleotide and a second sequence portion for hybridisation with the second oligonucleotide. The first detectable probe and the second detectable probe hybridize preferably between the first and the second sequence portion for hybridisation with the oligonucleotides.

The term “hybridisation” refers to the formation of a double stranded nucleic acid molecule consisting of a template nucleic acid and an oligonucleotide or a probe under both stringent and non-stringent conditions, whatever is suited for the analysis method.

It is preferred that the first sequence portion of the nucleic acid to be detected and/or of the competitive nucleic acid contains a sequence that is reverse complementary to the sequence of the first oligonucleotide and/or that the second sequence portion of the nucleic acid to be detected and/or of the competitive nucleic acid contains a sequence that is reverse complementary to the sequence of the second oligonucleotide. In the most preferred embodiment, the oligonucleotides serving as primers for the amplification reaction hybridize perfectly, e.g. without mismatch, with their respective template molecules.

In other words, it is preferred that the first sequence portion of the nucleic acid to be detected and the first sequence portion of the competitor nucleic acid are identical, and/or that the second sequence portion of the nucleic acid to be detected and the second sequence portion of the competitor nucleic acid are also identical.

Besides using the same set of oligonucleotides as primers for both template molecules (e.g. the nucleic acid to be detected and the competitor nucleic acid), it is also preferred that the amplification product of the nucleic acid to be detected and of the competitor nucleic acid are essentially identical in length and of a similar composition of nucleic acid bases, which facilitates equal amplification efficiency in the amplification of the two template species. The term amplification product refers to the product of the amplification reaction obtained by using the first oligonucleotide and the second oligonucleotide. The length of the two amplification products differs preferably less than 15%, most preferably less than 5%. This length difference between the amplification product of the nucleic acid to be analyzed and the competitor nucleic acid is preferably located in the sequence portion of the amplification products to which the respective probe hybridizes to, and/or to the portion that at least one of the oligonucleotides hybridizes with. Nonetheless, differences in length between target nucleic acid and competitor nucleic acid may be acceptable if both nucleic acids result in amplification of comparable efficiencies.

It is further preferred that the amplification product of the nucleic acid to be detected and the amplification product of the competitive nucleic acid differ in sequence in 1 to 20 nucleotides (nt), or up to 15%.

It is most preferred that the nucleic acid to be detected and the competitor nucleic acid differ in their sequence only at a sequence portion where the detectable probe hybridizes. Therefore, the first detectable probe and the second detectable probe differ in one embodiment only in one position in the sequences, since this is sufficient under stringent amplification conditions to differentiate between the amplification products. This further contributes to identical amplification efficiency for both template molecules. In another embodiment, the first detectable probe and the second detectable probe differ in between 30 and 2 positions in their sequences.

The step of determining the quantitative amount is preferably performed by comparing the position of the signal stemming from the first probe with the position of the signal stemming from the second probe.

The amount of competitor nucleic acid molecules can either be known as absolute copy numbers or can be determined empirically through a serial dilution of a competitor stock solution of unknown concentration. In a preferred embodiment a calibration is done as follows: The competitor nucleic acid is added to a constant amount of sample matrix that contains the organic substance at a definite amount, such as an analytical threshold, is co-extracted with the nucleic acid to be detected, amplified and detected in a way that both signals, of the nucleic acid to be detected and of the competitor nucleic acid, appear at the same position (threshold cycle) in a real-time PCR. According to this procedure, the competitive real-time PCR with sequence specific primers and fluorescence probes is calibrated to a desired amount of the species to be detected and quantified without necessarily knowing the quantitative number of amplifiable copies of the nucleic acid to be detected. This way, the calibration can be done for a given amount of species to be detected in a sample matrix, such as a threshold provided by law for the presence of an allergenic food ingredient to be labelled.

As an example, the calibration with competitor can be based on a food sample that contains an allergenic ingredient, such as whole peanut, in an amount that makes labelling the ingredient legally obligatory. The amount of competitor nucleic acid necessary needs to be determined empirically, as mentioned above. The determined amount of competitor nucleic acid to achieve essentially identical or similar signal positions for both the nucleic acid to be detected and the competitor nucleic acid is added to a given amount of sample that contains an unknown amount of the ingredient to be detected and quantified. In other words, first an appropriate amount of competitor nucleic acid needs to be determined that yields an appropriate signal in a suitable amplification reaction. Then, this appropriate amount is added to the sample of unknown amount of organic substance as an internal standard. The amount of competitor nucleic acid is chosen such as to represent the threshold value of the organic substance in the sample.

If both signals, from the nucleic acid of the ingredient to be detected and of the competitor nucleic acid, appear at the same threshold cycle, it can be concluded that the analyzed sample matrix contains the ingredient species to be detected in an amount equal to the level the method was calibrated for using the competitor nucleic acid. If the competitor signal appears later in a real-time PCR, i.e. at a higher C_{T} value, than the signal of the nucleic acid to be detected (the target species), it can be concluded that the sample matrix contains more of the target species than the amount the method was calibrated for with the competitor nucleic acid. Vice versa, if the competitor signal appears earlier in a real-time PCR, i.e. at a lower C_{T} value, than the signal of the sample matrix, the sample matrix does contain less of the target species than the calibration level. Thus, absolute quantification is achieved at the level of calibration.

Moreover, a semi-quantitative conclusion can be drawn if the positions of the signal from the competitor nucleic acid and of the nucleic acid to be detected differ from each other. Specifically, it can be concluded that the amount of nucleic acid to be detected in the sample is “higher” or “lower” than the amount that the threshold value stands for.

If the efficiency of both the amplification, e.g. the real-time PCR, and the hybridization reactions of the first and the second detectable probes are identical and around 100%, the absolute difference in threshold cycles between both targets can be used to calculate the amount of species in the sample as a factor in comparison to the calibrated level and without further need for efficiency control. Then, a difference of 3.332 cycles between the real-time PCR signals forms the basis of a factor that represents the difference in the amount of the nucleic acid to be detected relative to the calibrated threshold value. Specifically, a difference of 3.332 amplification cycles between the signal from the nucleic acid to be detected (e.g. peanut) and from the competitor nucleic acid (with the position of the signal from the nucleic acid to be detected being lower) would allow the conclusion that the amount of peanut is 10 times higher in the sample than the amount that is represented by the calibrated analytical threshold value.

It is furthermore possible to use an external standard for the nucleic acid to be detected and/or for the competitor nucleic acid, to calculate the ratio between the amplicons of the nucleic acid to be detected and of the competitor nucleic acid, and to relate this ratio to the calibrated threshold of organic substance.

The method according to the invention is therefore preferably an absolute quantitative detection method. It is preferred that first detectable probe and the second detectable probe are each tagged with a detectable label. Such a label can be for example FAM, HEX, Cy3 or Cy5. In order to distinguish between the probes, the detectable label of the first detectable probe is distinguishable from the detectable label of the second detectable probe.

It is preferred that the detectable label is suitable for a real-time PCR assay. The probe can be modified, for example with a quencher and/or a label for detection as known by a person of skill in the art, like the dye FAM or the quencher BHQ black hole or dabcyl.

The problem underlying the present invention is also solved by the use of a method described above and herein for the quantitative detection of an organic substance in a sample, based on its nucleic acid, in particular for the quantitative detection of food allergens, bacteria, viruses, mutations (like SNPs) in a sample. The sample can be a food sample, a tissue, an organ, and a body fluid. The term body fluid is meant to comprise fluids such as whole blood, blood plasma, blood serum, urine, sputum, tears, sweat, saliva, lymph fluid, liquor or alike. In such a use, a sample containing a nucleic acid is provided.

The problem underlying the present invention is also solved by a packaging unit or kit according to the claims. Such a kit comprises at least three types of vessels vessels, each comprising different ingredients. The kit may be stored over extended periods of time without loss of activity due to the fact that the ingredients are rendered durable by them being provided inside the reaction vessels. Durability of the kit may be further improved if the contents of the vessels are supplied inside in solution phase, as materials reversibly bound to an enclosed solid phase to be added, or in one or more layers of trehalose dried onto the inner surfaces of the reaction vessels.

The different sets vessels fulfill the following roles: (i) The at least one vessel comprising defined amounts of target and competitor nucleic acid is used for a one-point calibration of the real-time PCR instrument. The amount of nucleic acid to be detected in the calibration vessel is set to a desired threshold value, such as a value corresponding to the maximum legal concentration of a compound in a standardized food sample. (ii) The at least one vessel for extraction of the target nucleic acid from the sample, each comprise a threshold-calibrated normalised amount of competitor nucleic acid. The amount of competitor nucleic acid is preferably identical to that used in the vessel (i). The user introduces a pre-defined amount of sample into this vessel and uses it for the extraction of nucleic acid. Hence he will obtain an extraction mixture of nucleic acids from the sample (including the nucleic acid to be detected) and of a standardized amount of competitor nucleic acid. (iii) The one or more vessels for performing real-time PCR each preferably comprise standardised amounts of first and second primers and probes for calibrated real-time PCR of the target and competitor nucleic acids. An extraction sample from a vessel from (ii), comprising nucleic acid to be detected, as well as competitor nucleic acid is introduced into a sample (iii) and real-time PCR is performed. Since the target nucleic acid and the competitor nucleic acid are present in a set ratio (independent of the amount of extraction sample transferred from (ii), the amount of target nucleic acid may be determined compared to the amount of competitor nucleic acid, based on the relative C_{T} values obtained.

The target and competitor nucleic acids, and/or primers are preferably present in the vessels in solution phase, as materials reversibly bound to an enclosed solid phase to be added, or inside trehalose layers dried onto the inner surfaces of the vessels.

In one embodiment of the invention, to packaging unit or kit is suitable for the simultaneous determination and quantification of several nucleic acids. It comprises a calibration vessel with standardised threshold amounts of all the target nucleic acids and corresponding competitor nucleic acids, extraction vessels with standardised amounts of all the competitor nucleic acids and PCR-vessels with primers for the amplification of all target and competitor nucleic acids. The probes are preferably labelled with markers or labels ensuring that all the amplified target and competitor nucleic acids may be separately detected.

The method and packaging unit or kit of the present invention may, among others, be used for the detection and quantification of the following foodstuffs, products thereof and traces thereof: gluten, crustaceans, eggs, fish, peanuts, soybeans, milk, nuts (including almonds, hazelnuts, walnuts, cashews, pecan nuts, Brazil nuts, pistachio nuts, macadamia nuts, queensland nuts), celery, mustard, sesame seeds, lupin, molluscs. Other possible targets are genetically modified organisms such as Roundup Ready Soy, or MON810 maize, animal species (pork, beef, chicken, fish etc.), pathogens in tap water, bottled water, or foodstuffs (e.g. salmonella, listeria, novovirus etc.), spoilage agents (yeasts, fungi etc.), parasites or bacteria in stool samples (e.g. Giardia lamdlia, Cryptosporidiae, Entamoeba, Microsporidiae, salmonella etc.), viruses in stool or tissue samples (e.g. Norovirus, Astrovirus, Rotavirus etc.) and so on. The method and kit may further be used for the detection and quantification of any RNA or DNA-virus in human tissue or liquid samples.

The method according to the present invention is based on a competitive Polymerase Chain Reaction setting to quantify an organic substance. Since the underlying example is performed in a real-time PCR cycler environment, this invention is called a calibrated real-time cqPCR.

Further problems, advantages and solutions of the inventions may be derived from the subsequent examples and the figures:

### BRIEF DESCRIPTION OF THE FIGURES

FIG. 1: generation of a 107 bp competitive nucleic acid in the form of a competitive double-strand DNA with conserved primer binding sites but a divergent binding site for detectable probes for discriminating between the 86 bp long peanut-specific PCR product and the competitive nucleic acid; the overall nucleotide composition is nearly conserved between the two amplifiable sequences; the melting temperatures differ only by 2 K; all DNA sequences are displayed from left to right from the 5′ to the 3′ end;

FIG. 2: purified 107 bp competitive DNA at different amounts (lanes 1-3) in comparison to 100 bp DNA ladder (lane M) in agarose gel electrophoresis;

FIG. 3: detection of the PCR-products of a competitive PCR in agarose gel electrophoresis (M, size marker; N, no template control); copy numbers of amplified 107 bp competitor nucleic acid and 86 bp nucleic acid to be detected in the form of peanut DNA are shown as orders of approximate magnitude;

FIG. 4: comparable efficiencies of nearby 100% for both the amplification of the peanut-DNA (nucleic acid to be detected; FAM reporter dye: blue filled squares) and the competitor-DNA (competitor nucleic acid; HEX reporter dye, green filled squares);

FIG. 5: primary titration of competitor copies versus chocolate that contains peanut (nucleic acid to be detected) at the 100 ppm level (75.9 ppm);

FIG. 6: final titration of competitor copies versus chocolate that contains peanut (nucleic acid to be detected) at the 100 ppm level (75.9 ppm).

### EXAMPLE 1

Extraction of Nucleic Acid in the Form of DNA from Food.

A representative sample was ground to homogeneity with an analytical mill (IKA M20, IKA Labortechnik, Staufen, Germany). Of the homogenized sample, exactly 300 mg (+/−15 mg) were transferred to a 2 mL microreaction tube and suspended in 1.5 mL of CTAB buffer (55 mmol/L hexadecyltrimethyl-ammoniumbromide (CTAB), 1400 mmol/L NaCl, 20 mmol/L ethylenediamintetraacetate (EDTA), 100 mmol/L TRIS (tris(hydroxymethyl)-aminomethane), pH 8.0, adjusted with 10% HCl, autoclaved). Extraction was carried out in a thermomixer (no. 5437, Eppendorf, Hamburg) at 65° C., 1000 rpm, for 5 min or until a homogeneous suspension was achieved. After addition of 20 μL of 20 mg/mL proteinase K (Merck, no. 1.24853, DNA grade, Darmstadt, Germany), the sample was digested at 65° C., 1000 rpm, for 30 min. For removal of undissolved material, the tube was centrifuged at 14,000 rpm for 2 min in a common desk top centrifuge for microtubes. Subsequently, digested proteins and Proteinase K were removed by chloroform extraction. Therefore, 800 μL of the supernatant were transferred to a 2 mL microreaction tube and 600 μL of chloroform were added. Peptides were precipitated by strong agitation on a vortexer. The tube was centrifuged as described above and 600 μL of the DNA-containing upper phase were transferred to another 1.5 mL microreaction tube. After addition of 1 μL of 20 mg/mL glycogen (Roche, no. 10901393001, for molecular biology) and 500 μL of 80% isopropanol, the DNA was precipitated by strong agitation on a vortexer for 30 sec. The precipitate was centrifuged as described above and the supernatant was discarded. The resulting DNA pellet was washed once by addition of 500 μL of 70% ethanol and the ethanol was removed. The pellet was dried at 50° C. for 5 min in the thermomixer. The dried pellet was dissolved at 4° C. overnight in 100 μL of TE buffer (10 mmol/l TRIS, 1 mMol/L EDTA, pH 8.5, adjusted with 10% HCl, autoclaved) or as much as need to fully dissolve the pellet.

100 μL of the extracted DNA was further purified by use of the QIAquick PCR Purification Kit (Qiagen, Hilden, no. 28106) according to the protocol using a micro-centrifuge. Briefly, 500 μL of buffer PB were added to 100 μl of extracted DNA and the protocol was strictly followed according to the manufacturer's instructions. The purified DNA was eluted by 50 μL of buffer EB from the column. The column was once more eluted with 50 mL of the first elution. The purified DNA was subjected to PCR analysis or stored at −20° C. until use.

### EXAMPLE 2

### Calibration of Nucleic Acid Extraction by the Addition of Competitor Nucleic Acid

The DNA-extraction was performed identical as described above except for the addition of competitor nucleic acid as internal standard. Prior to the addition of 20 μL of 20 mg/mL Proteinase K (Merck, no. 1.24853, DNA grade, Darmstadt, Germany), the titrated amount of competitor nucleic acid was pipetted in at total volume of 7.5 μL of 10 mmol/L Tris-Cl, pH 8.5. After the addition of proteinase K, the protocol was again strictly followed as described above. Serial quantitative analysis of food samples on the basis of nucleic acids is difficult and error-prone because, while the extraction of nucleic acids is a standard procedure, the yield of the nucleic acid is by no means constant but very much dependent on the type of matrix and the kind of food. More precisely, when analysing different kinds of chocolate (e.g. bitter chocolate, milk chocolate, hazelnut chocolate, marzipan chocolate and nougat) the yield or amount of nucleic acid extracted will depend very much on the type of matrix. Thus, it is necessary to calibrate nucleic acid extraction for each sample, and this is best done according to the invention by adding, prior nucleic acid extraction, a defined amount of nucleic acid which will serve as competitor nucleic acid within subsequent cqPCR analysis. Such a calibration of nucleic acid extraction is in particular helpful in case of food or otherwise processed samples, and other samples subjected to a typical mechanical shearing of the nucleic acids for breaking up the sample matrix, because the length of the nucleic acid fragments in such mechanically processed samples corresponds very much to the length of the PCR-amplicons, typically between 50 to 250 bp. In essence, the competitor nucleic acids will perform similarly or identically in the extraction as nucleic acids from the sample matrix.

While the total amount of extracted nucleic acids will still vary depending on the type of the extracted matrix, the ratio of target nucleic acids of the sample matrix to competitor nucleic acid will stay the same throughout the entire nucleic acid extraction procedure. When the competitor nucleic acid is added such that it represents the threshold ratio (the amount of allowable target organic species compared to the total weight of the sample), the nucleic acid extraction does no longer affect and interfere with a quantitative analysis by a calibrated cqPCR based on a threshold as the yield of extracted nucleic acid does not affect the ratio of target nucleic acid to total nucleic acids in the sample.

### EXAMPLE 3

### First and Second Oligonucleotides, First and Second Detectable Probes

The peanut specific primers and probes for genomic peanut DNA and nucleic acid competitor as well as the synthetic oligonucleotides for the generation of competitor dsDNA were commercially synthesized by commercial suppliers (Biomers, Ulm, Germany; and Biotez, Berlin, Germany). All oligonucleotides are displayed from left to right from the 5′ to the 3′ end. The hybridizing ends of the competitor synthesis oligonucleotides are underlined.

Forward primer:
(SEQ ID NO 1)
GCAGCAGTGGGAACTCCAAGGAGACA
Reverse primer:
(SEQ ID NO 2)
GCATGAGATGTTGCTCGCAG
Peanut DNA specific probe:
(SEQ ID NO 3)
FAM-CGAGAGGGCGAACCTGAGGCC-BHQ
Competitor DNA specific probe:
(SEQ ID NO 4)
HEX-CCGGAGTCCAAGCGGGAGAGC-BHQ
Synthesis oligo #1:
(SEQ ID NO 5)
GCAGCAGTGGGAACTCCAAGGAGACAGAAGATGCCAGAGCCAGCTGCAAG
GGCGGCCGGAGTCCAAGCGGGAGAGC
Synthesis oligo #2:
(SEQ ID NO 6)
GCATGAGATGTTGCTCGCAGGCGCCTAGTGTGCTCTCCCGCTTGGACTCC
GG

It is obvious to a person skilled in the art that other sample and other analysis will require other primers while the basic principles of the method and kit of the invention will stay the same.

### EXAMPLE 4

### Generation of Competitor Nucleic Acid for Addition to the Sample

First, each 50 pmol of the synthesis oligos #1 and #2 were mixed in 20 μL of 1× PCR buffer (without MgCl₂), heated at 96° C. for 30 sec, and cooled to ambient temperature for hybridization. Second, 2 μL of the hybridization mixture were added to a total volume of 20 μL of 1× Klenow buffer (10 mmol/L Tris-CL, 5 mmol/L MgCl₂, 3.75 mmol/L DDT, pH 7.5) that contains 4 Units of Klenow Fragment (Roche Applied Science, #11008404001) and 0.1 mmol/L of each dNTP (dATP, dTTP, dGTP, and dCTP). After incubation at 37° C. for 30 min, the 20 μL of Klenow reaction mixture was purified with a Centri Spin 20 column (Princeton separation, #CS-200, via EMP Biotech GmbH, Berlin) according to the manufacturer's instructions. Then, 5 μL of purified competitor nucleic acid were added to a total volume of 50 μL PCR reaction mix that contains 1× PCR buffer A, 800 μM dNTP mix (each 200 μM dATP, dTTP, dGTP, and dCTP), 2 mmol/L MgCl₂, and 1.25 Units of Platinum® Taq (Invitrogen, Karlsruhe, Germany). PCR was performed with 40 cycles of 30 sec at 95° C., 30 sec at 60° C. and 30° sec at 72° C. The 107 bp competitor PCR product was purified with a Centri Spin 20 column. The quality of the purified competitor DNA was investigated by agarose gel electrophoresis with subsequent ethidium bromide fluorescence staining: 5 μL of PCR product and 1 μL of 6× gel loading concentrate (15% Ficoll™, 0.25% bromophenol blue, 0.25% xylenecyanol in distilled water) were mixed, and PCR products separated on 3% agarose (Carl Roth, no. K 297.2, Karlsruhe, Germany) gels by rapid agarose gel electrophoresis (RAGE) (Cascade Biologics, via TEBU, Frankfurt/M, Germany) according to the manufacturer's instructions. Thereafter, gels were soaked in 0.5 μg/mL of ethidium bromide for 20 min. Thereafter, PCR products were visualized on a UV-transilluminator (TFM20, UVP, via Merck). The size of the PCR-products was controlled by comparison with a 100 bp size marker (Gen-Sura, SLL 101, via Carl Roth) and documentation of results was done with an instant camera (Polaroid GelCam, film type 667, ¼ sec, 5.6f).

For determination of the concentration of genomic DNA, 5 μL of DNA was measured at 260 nm wavelength in a microcuvette in a spectrophotometer. One OD of dsDNA at 260 nm was equivalent to 50 ng/μL of genomic DNA. From the determined amount of competitor DNA, the copy number was calculated considering the nucleic acid composition and length of the competitor dsDNA.

The competitor DNA PCR product was serially diluted in TE buffer (10 mmol/L TRIS, 1 mmol/L EDTA, pH 8.5) containing 0.1% Tween 20, and stored at −20° C. until use.

### EXAMPLE 5

### Generation of the Purified Peanut DNA Amplicon

Genomic DNA was purified from peanut as described above. As described above for the competitor DNA, the PCR was performed with peanut genomic DNA to generate an 86 bp peanut PCR product. The peanut PCR product was further purified, quantified by UV spectrophotometer, serially diluted, and stored as described above.

### EXAMPLE 6

Real-Time cqPCR and Detection of Amplicons from Peanut DNA (Nucleic Acid to be Detected) and Competitor DNA (Competitor Nucleic Acid)

Polymerase chain reaction of peanut DNA in the presence or absence of competitor DNA was carried out in 0.2 mL thin-walled micro-tubes (Advanced Biotechnologies, Hamburg, Germany) at final volumes of 50 μL. The PCR mixture contained 1× reaction buffer (20 mmol/L Tris HCl (pH 8.4), 50 mmol/L KCl; supplied with Platinum® Taq, Invitrogen, Karlsruhe, Germany), 5.0 mmol/L magnesium chloride (supplied with Platinum® Taq, Invitrogen); 200 μM each of the deoxyribonucleoside triphosphates (dNTPs) dATP, dCTP and dGTP, and 400 μM of dUTP as substitute for dTTP, and derived from a dUTP-containing dNTP mix (Carl Roth, Karlsruhe) where A is adenosine, C is cytidine, G is guanosine, U is uracil, and T is thymidin; 0.25 μg/μL of bovine serum albumin (Sigma, no. A 8022, Deisenhofen, Germany); 300 nmol/L of each primer ‘forward’ and ‘reverse’; 200 nmol/L labelled peanut probe; 200 nmol/L labelled competitor probe; 0.5 units of uracil-N-glycosilase (AmpErase UNG, PE Biosystems, no. N 808-0096); 1.25 units of Platinum® Taq Gold polymerase (Invitrogen); and 1/10 of the reaction volume of undiluted extracted DNA (5 μL). The PCR was carried out in an Mx3005P™ QPCR system (Stratagene, via Agilent Technologies Sales & Services GmbH & Co. KG, Waldbronn, Germany) using the following conditions: chemical decontamination reaction from dUTP-containing template at 50° C. for 2 min, deactivation of UNG and activation of Platinum® Taq Gold polymerase at 95° C. for 2 min, followed by 45 two-step cycles with a DNA denaturation at 95° C. for 30 sec, and combined primer annealing and elongation at 62° C. for 30 sec. Fluorescence was recorded after each cycle at both the FAM and HEX channel.

### EXAMPLE 7

Preparation of Chocolate Spiked with Peanut at Defined Levels

Milk chocolate containing 100 ppm (0.01%) peanut was prepared similar as described for hazelnut (Holzhauser T, Vieths S. Quantitative sandwich ELISA for determination of traces of hazelnut (Corylus avellana) protein in complex food matrices. J. Agric. Food Chem. 1999, 47: 4209-4218): Peanut-free chocolate (prechecked by peanut-specific in-house ELISA and real-time PCR) was spiked with finely ground peanut cream, that consisted of 100% peanut (unknown variety) from a commercial retailer (GranoVita, #1008) at levels of 10%, 1%, 0.1%, 0.01%, and 0.001%. 1 g of peanut was added to 9 g of a food sample, and the spiked sample was melted at 37° C. and mixed with a stirrer for better sample homogeneity. Thereafter, 1 g of the spiked sample containing 10% of peanut was added to another 9 g of chocolate. The procedure was continued until a sample containing 0.001% of peanut was obtained.

The accuracy of the spike levels in milk chocolate was verified with the in-house peanut specific ELISA in comparison to the peanut reference material ‘peanut cream GranoVita’ as was previously described for hazelnut (Holzhauser T, Vieths S. Quantitative sandwich ELISA for determination of traces of hazelnut (Corylus avellana) protein in complex food matrices, J. Agric. Food Chem. 1999, 47: 4209-4218).

Each of the samples containing peanut at the concentration levels described was extracted according to the DNA extraction protocol for PCR-analysis and according to protein extraction protocols for ELISA analysis.

### EXAMPLE 8

Samples with Defined Amount of Peanut from Official Ring Trials

A milk chocolate with 2 ppm peanut, a dark chocolate with 20 ppm peanut, and a milk chocolate with 100 ppm peanut previously employed in a ring trial for performance testing as official German food regulatory method according to §64 of the German “Lebensmittel-, Bedarfsgegenstände- and Futtermittelgesetzbuch” (LFGB) were included in this study. Each of the samples was extracted according to the DNA extraction protocol for PCR analysis and according to protein extraction protocols for ELISA analysis.

### Commercial Food Samples

Commercial food samples were purchased from a local retailer.

### EXAMPLE 9

Calibration of Real-Time cqPCR for Peanut Quantification

The competitor DNA was serially diluted to allow addition to the self prepared chocolate prior to DNA extraction. Identical portions of 300 mg chocolate, containing peanut at the 100 ppm level (exactly 75.9 ppm), were fortified with increasing amounts of competitor DNA. After DNA-extraction, the FAM (peanut DNA probe) and HEX (competitor DNA probe) signals were recorded in real-time PCR allowing to determine the threshold cycles for the FAM and HEX signal at pre-defined fluorescence intensity. The amount of copy number of peanut DNA and competitor DNA was quantified against an external standard curve of peanut DNA PCR product and an external standard curve of competitor DNA, respectively. The logarithm of the copy ratio (peanut DNA/competitor DNA) was plotted against the logarithm of competitor DNA copies. After linear regression, the copy number necessary for equivalence of the copy numbers of peanut and competitor DNA was calculated from the linear equation.

### EXAMPLE 10

Semi-Quantitative Evaluation and Quantitative Determination by Real-Time Calibrated cqPCR

The real-time cq-PCR was calibrated to match 100 ppm (0.01%) peanut in a chocolate sample. The semi-quantitative analysis allows to distinguish between the three cases A) peanut (FAM) and competitor (HEX) specific signal arise at identical threshold cycle (C_{T}): the sample contains 0.01% peanut, B) C_{T} (FAM)>C_{T} (HEX): the sample contains more than 0.01% peanut; and C) C_{T} (FAM)<C_{T} (HEX): the sample contains less than 0.01% peanut. At the level of equivalence of both peanut and competitor specific signal, the detection is considered quantitative. Divergent signals allow semi-quantitative evaluation of the result.

Moreover, calculation of the amount of peanut in the sample can be done based on the following equation, provided that the efficiency of both the amplification of peanut DNA and competitor DNA is at or nearby 100%:

### Here:

$$\begin{matrix}\% peanut in sample = {2}^{Δ CT} \cdot ( \% level of calibration ) \\ = {2}^{[ CT ( competitor ] - CT ( peanut ) ]} \cdot ( \% level of calibration ) ;\end{matrix}$$

$$\% peanut in sample = 0.01 \cdot {2}^{CT ( HEX ) - CT ( FAM )}$$

### COMPARATIVE EXAMPLE 11

### In-House-ELISA for the Quantification of Peanut

The sandwich-type ELISA for peanut quantification including protein extraction from food samples was performed as previously described (Stephan O, Vieths S. Development of a real-time PCR and a sandwich ELISA for detection of potentially allergenic trace amounts of peanut (Arachis hypogaea) in processed foods; J. Agric. Food Chem. 2004, 52: 3754-60). The limit of detection is 1 ppm (mg/kg) peanut.

### Commercial ELISA for the Quantification of Peanut

Three commercial peanut specific ELISAs, all certified as ‘AOAC Research Institute Performance Tested Method’, were included to compare to the quantitative peanut-PCR in this study: BioKits Peanut Assay Kit (Tepnel BioSystems, via Coring Sytem Diagnostix GmbH, Gernsheim, Germany), RIDASCREEN® FAST Peanut (R-Biopharm AG, Darmstadt, Germany), and NEOGEN Veratox® Peanut Allergen Test (Neogen Europe Ltd., Auchincruive, Ayr, Scottland). All three commercial ELISAs feature peanut standards for direct read-out as ‘peanut’ without conversion from detectable peanut protein to peanut. The extraction buffers and protocols were included. The commercial ELISAs including sample preparation were performed according to the manufacturers' instructions.

### EXAMPLE 12

Analysis of Results with Various Peanut Spiked Milk Chocolates as a Sample

Peanut spikes were prepared as described in Example 7. The accuracy of the spikes was verified by the in-house peanut specific ELISA. This ELISA quantifies peanut protein. To determine the amount of peanut, quantified peanut protein is multiplied by a factor of conversion. For exact determination of peanut in a sample, the peanut to be quantified needs to be available as a reference. First, peanut protein from peanut cream ‘GranoVita’ was quantified by the in-house peanut specific ELISA. The peanut cream had 29.15% extractable and quantifiable peanut protein, resulting in a conversion factor of 3.43 to calculate the amount of peanut from quantified peanut protein in the spiked milk chocolate samples. The results of the quantified peanut in the spikes of milk chocolate are displayed in Table 1.

### Optimization of the Peanut Specific PCR

The amplification of an 86 bp peanut specific PCR product is based on the Ara h 2 gene (NCBI GenBank acc no. L77197). The method for amplification of peanut specific DNA was in principle described previously (Stephan O, Vieths S. Development of a real-time PCR and a sandwich ELISA for detection of potentially allergenic trace amounts of peanut (Arachis hypogaea) in processed foods; J. Agric. Food Chem. 2004, 52: 3754-60). However, one primer mismatch was corrected and the PCR performance was optimized to obtain a PCR efficiency of almost 100%. This was achieved by additionally changing the chemistry of the PCR reaction mixture including the hot start polymerase and the parameters of thermal cycling the PCR. The PCR was further performed as described herein.

Generation of a Competitor dsDNA

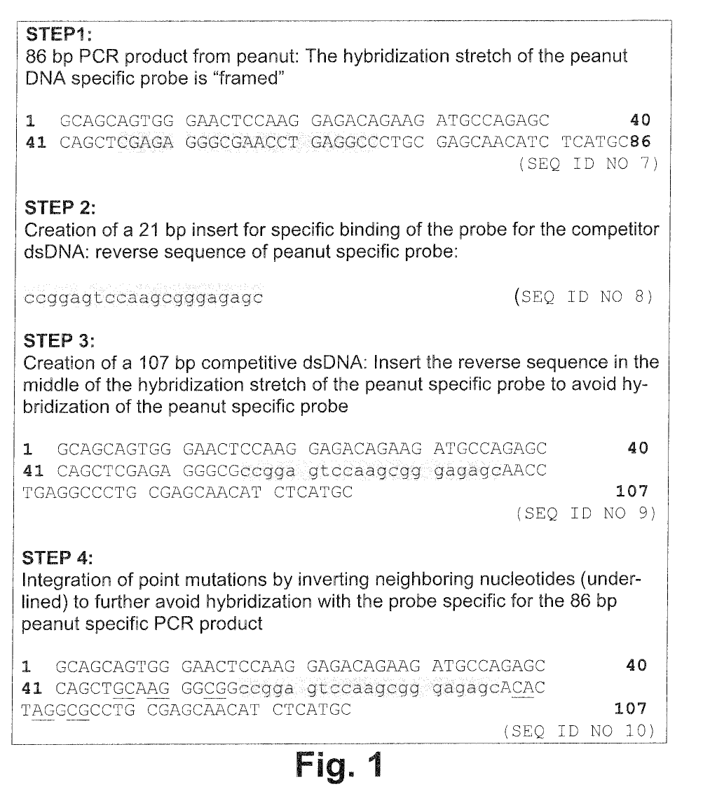

The 107 bp double-stranded competitor DNA was generated by the use of two overlapping synthetic oligonucleotides that were hybridized, the 3′ ends filled up with Klenow fragment that lacks the 5′→3′ exonuclease activity of the native enzyme, and finally amplified in PCR with the peanut DNA specific primers and Taq Polymerase. The underlying strategy to generate a competitive DNA with conserved primer binding sites but divergent probe hybridization stretches as well comparable composition of total nucleotides to allow differentiation of PCR products from peanut genomic DNA and competitor DNA by A) size for analysis in electrophoresis post PCR, and B) specific probes for real-time detection in PCR is given in FIG. 1.

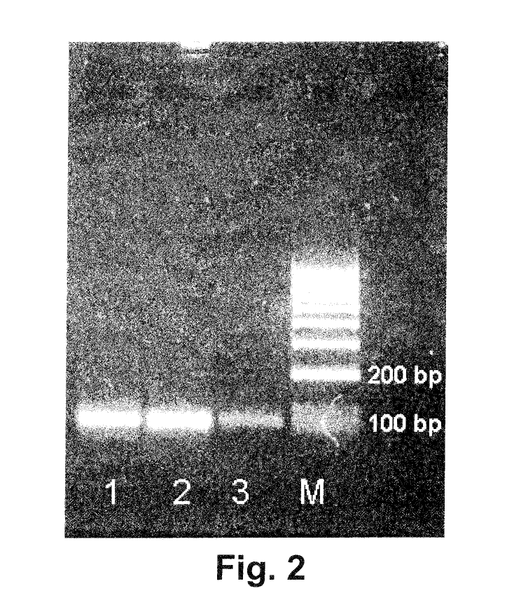

The quality of the purified competitor DNA PCR product was evaluated by agarose gel electrophoresis and fluorescence staining with ethidium bromide: By visual inspection, the competitor DNA PCR product comprised only one single DNA band of appropriate length (FIG. 2). The serially diluted 107 bp competitor DNA was stored at −20° C. until use.

### Classic Competitive PCR

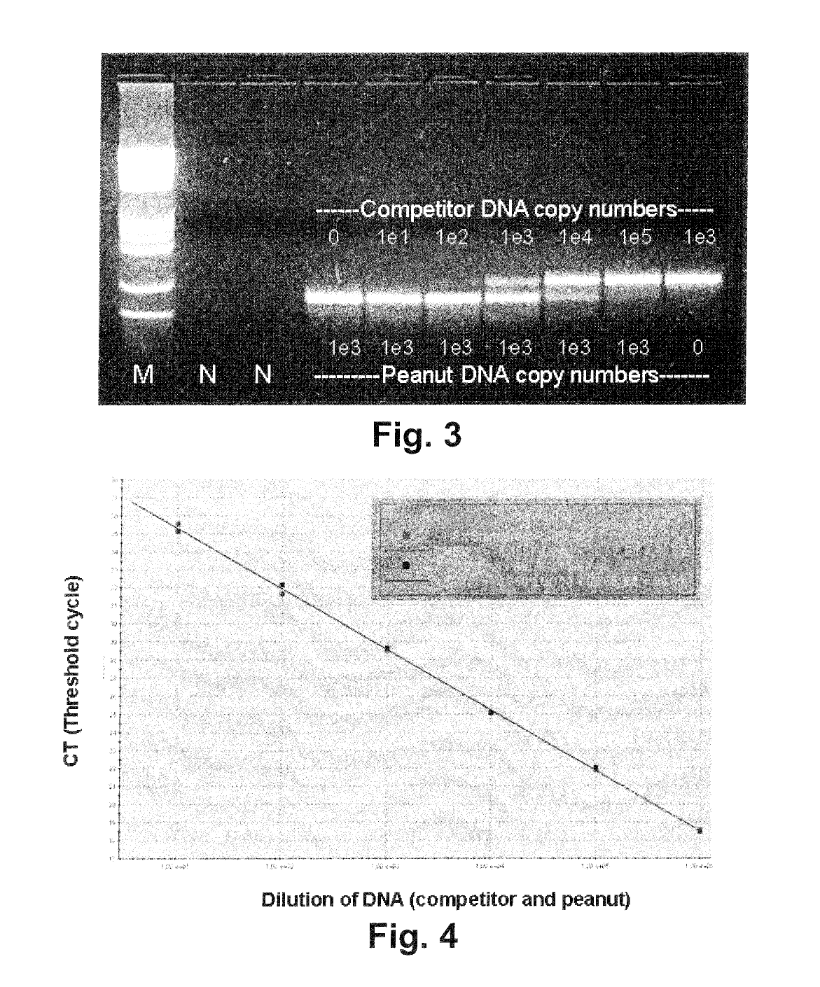

A classic competitive PCR with post-PCR agarose gel electrophoresis was run with a stock dilution of the 86 bp peanut DNA PCR-product corresponding to approximately 1E3 copies, and increasing copies of 107 bp competitor for demonstrating the competitive reaction visually (FIG. 3). Obviously, PCR products of competitor and target (peanut) can be discriminated visually. However, determining the exact amount of competitor DNA to achieve identical amplification as for peanut DNA is difficult to achieve. Consequently, the competitive PCR was further set up for detection in real-time PCR.

### PCR Efficiency and Specificity for the Peanut Target DNA and Competitor DNA

Serial dilutions of peanut DNA PCR product were amplified in a non-competitive set-up, and a standard curve was recorded by plotting the C_{T} of each standard versus the logarithm of the copy number of amplifiable DNA, both for peanut DNA and competitor DNA. The efficiency is 100% at a slope of −3.322.

FIG. 4 displays complete overlay of the standard curves originating from peanut DNA (filled squares) and competitor DNA (filled circles) with identical PCR efficiency of nearby 100%.

Since both probes for peanut-DNA and competitor DNA are present in the PCR reaction mix, fluorescence cross-talk of the sequence specific probes were measured for the FAM-labelled probe with competitor DNA, and for the HEX-labelled probe with peanut DNA. No fluorescence cross-talk between fluorescence labels and no false priming of the probes was observed.

### EXAMPLE 13

### Calibration of Competitive Real-Time PCR for a Threshold of 100 ppm Peanut

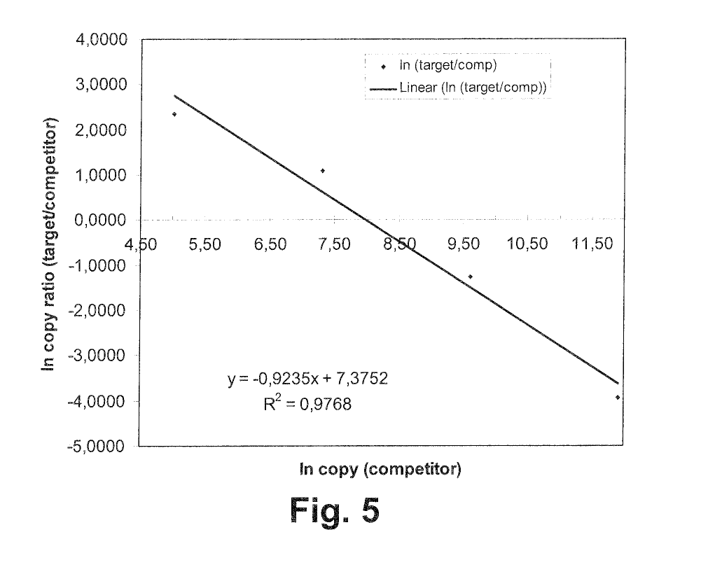

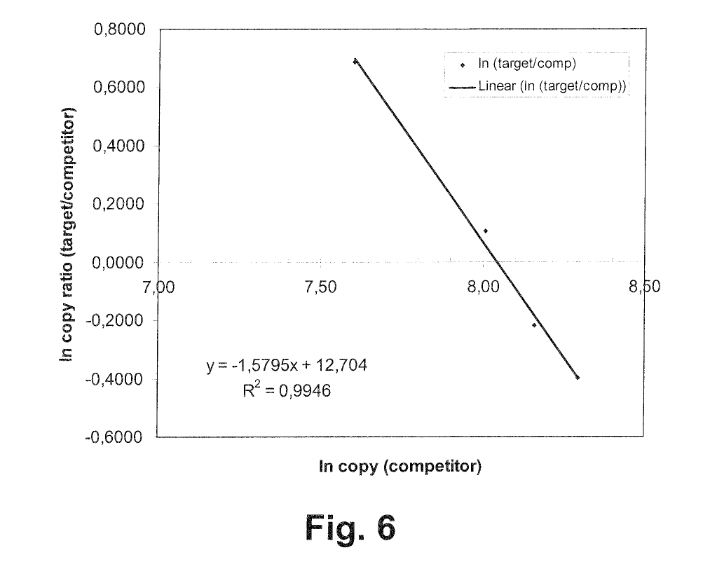

The milk chocolate spiked at the level of 100 ppm (0.01%) peanut was used as reference for calibration of the competitive real-time PCR. As a first step, a rough titration with competitor DNA between 150 and 150,000 copies was done to identify the order of magnitude of competitor copies that are necessary for equal amplification in the presence of DNA originating from 100 ppm peanut. The calibration plot is given in FIG. 5. Resolution of the equation of the linear regression for ‘y=0’ resulted in 2940 copies of competitor. As the second step, a more narrow range of competitor DNA between 2000 and 4000 copies was chosen. The resolution of the equation of the linear regression re-suited in 3112 copies of competitor DNA to compete equally with 100 ppm peanut (FIG. 6). Since the true amount of peanut in the chocolate sample is 75.9 ppm, a total of 4100 copies are necessary to compete equally with exactly 100 ppm of peanut. For quantitative analysis of any unknown sample, 4100 copies of competitor DNA were added to each sample during the DNA extraction as described in Example 2.

### EXAMPLE 14

### Semi-Quantitative and Quantitative Determination of Peanut in Chocolate

Each of the self-made peanut spiked chocolates were extracted in duplicate, each extract was quantified in two separate PCR reactions. The quantification was done based on the assumption of 100% efficiency of the PCR for peanut and competitor DNA. The results are shown in Table 2: 988 ppm and 75.9 ppm peanut were recovered at 122 and 113%, respectively. The chocolate spiked with 6.86 ppm was slightly overestimated with a recovery of 187%. The chocolates having 8944 and 96237 ppm were clearly overestimated, demonstrating that the quantitative range of the method spans approximately two orders of magnitude with each one order of magnitude above and below the level of calibration.

### EXAMPLE 15

Comparison of Quantitative Results of Calibrated Real-Time cq-PCR and Commercial ELISA for Peanut in Chocolate

All investigations were performed in the year 2007 with the current versions of the commercial peanut specific ELISA. The ELISAs were performed according to the manufacturer's instructions: The quantification was each based on singlet determination of duplicate protein extractions. In PCR, each sample was extracted for DNA in duplicate, and each DNA extract was quantified in duplicate PCR reactions. The results are summarized in Table 3.

Dark and milk chocolate spikes that ranged from 6.8 to 988 ppm peanut were quantified in PCR with recoveries between 89% and 187%. 2 ppm were still detectable in 2/4 reactions with 81% recovery. At the level of calibration, the recovery in PCR was 101% and 113%, respectively. By contrast, ELISA recoveries ranged between 5% and 248%. Partially, peanut below 10 ppm was not detectable in ELISA, depending on the manufacturer. PCR also succeeded in quantifying peanut in matrix other than chocolate, such as nougat, without need for further calibration.

### EXAMPLE 16

Quantification of Peanut using the Threshold-Based Real-Time cqPCR with Only 1-Point Calibration Instead of External Standard Curves for the Peanut DNA and Competitor DNA using a Kit

The kit according to the invention was used in the quantification of peanut in milk chocolate: A peanut-negative whole milk chocolate from a local retailer was fortified with 0.01% roasted and ground peanut cream. The amount of competitor DNA to be added prior to extraction and amplification of peanut DNA was titrated to C_{T}-signal equivalence between peanut C_{T} (FAM) and competitor C_{T} (HEX) in competitive real-time PCR as described above.

Real-time PCR for titration of the given amount of competitor DNA was performed in parallel to an additional PCR reaction tube containing equivalent amounts of competitor DNA and peanut target DNA, such as 1000 copies each or defined dilutions of a stock from peanut amplicon and competitor amplicon, respectively. This additional mixture of equivalent amounts of peanut DNA and competitor-DNA was applied to adjust the peanut and competitor specific fluorescence signals of the real-time PCR device in a way that such fluorescence adjustment leads to identical peanut C_{T} (FAM) and competitor C_{T} (HEX). This mixture, called 1-point calibrator, is stored in identical ready-to-use and single-use calibration vessels (as part of a kit) for future quantification experiments. These calibration vessels may comprise various amounts of target and competitor DNA depending on the threshold concentration of peanut and sample size.

According to this procedure, a chocolate matrix is calibrated to a threshold of 0.01% peanut in the presence of a 1-point calibrator for the real-time PCR device. External standard curves for peanut DNA and competitor DNA were not applied but substituted by the 1-point calibrator.

Extraction vessels were supplied, comprising the required pre-determined amount of competitor DNA. These extraction vessels were adapted to hold a set amount of sample matrix, such as for example 50 mg, 500 mg, 1 g or 5 g of sample matrix. The amount of sample matrix used is identical to the amount of sample matrix that was applied for calibration of 0.01% peanut in chocolate matrix.

For the purpose of quantification, any matrix of interest was analyzed and peanut quantified under the performance of the following steps: First, the given amount of competitor-DNA was added to the defined amount of sample. Alternatively, the sample was introduced into an extraction vessel already comprising the given amount of competitor DNA. After extraction and purification of sample-DNA in the presence of the defined amount of added competitor-DNA, the extracted sample DNA was amplified in real-time competitive PCR. Second, the 1-point calibrator was amplified in parallel. The fluorescence signals of peanut DNA (FAM) and competitor DNA (HEX) of the 1-point calibrator were adjusted such as to achieve identical peanut C_{T} (FAM) and competitor C_{T} (HEX), respectively. Under this condition, the peanut C_{T} (FAM) and competitor C_{T} (HEX) of the sample were compared by either A) semi-quantitative evaluation or B) quantitative determination.

The amplification was carried out in pre-supplied real-time PCR vessels, already comprising the required amounts of primers, reagents and enzymes.

The semi-quantitative analysis allowed to distinguish between the three cases A) peanut (FAM) and competitor (HEX) specific signal arise at identical threshold cycle (C_{T}): the sample contained 0.01% peanut, B) C_{T} (FAM)>C_{T} (HEX): the sample contained more than 0.01% peanut, and C) C_{T} (FAM)<C_{T} (HEX): the sample contained less than 0.01% peanut. At the level of equivalence of both peanut and competitor specific signal, the detection was considered quantitative. Divergent signals allowed semi-quantitative evaluation of the result.

Moreover, calculation of the amount of peanut in the sample could be done based on the following equation, provided that the efficiency of both the amplification of peanut DNA and competitor DNA is at or nearby 100%:

$$\begin{matrix}\% peanut in sample = {2}^{Δ CT} \cdot ( \% level of calibration ) \\ = {2}^{[ CT ( competitor ) - CT ( peanut ) ]} \cdot ( \% level of calibration ) ;\end{matrix}$$

$$\% peanut in sample = 0.01 \cdot {2}^{CT ( HEX ) - CT ( FAM )}$$

### Quantification of 100 ppm Peanut in Various Food Matrices

Along with the self-prepared 100 ppm (0.01%) spike of peanut in milk chocolate, a self-prepared cookie dough with 100 ppm peanut as well as one milk chocolate and one dark chocolate with 100 ppm peanut were analyzed and quantified according to the above described method. The latter two samples were previously employed in a ring trial for performance testing as official German food regulatory method (according to §64 LFGB).

Each sample was extracted twice according to a given procedure. Each extract was quantified in duplicate calibrated cqPCR reactions according to the above described procedure. Hence, the results were means of 4 PCR reactions. The results are given in Table 4. Independent of the food matrix, the 1-point calibration allowed to quantify peanut in three different food matrices, namely milk chocolate, dark chocolate and cookie dough with recoveries between 74 and 102%, indicating high accuracy of the method. The quantitative variation between 4 PCR-reactions of an individual sample matrix ranged between 17 and 29%. This high reproducibility indicates that even a single extraction and a single amplification in real-time cqPCR can be done without substantial loss of quantitative characteristics.

Reproducibility of the Calibrated Real-Time cqPCR Step

The self-prepared 100 ppm (0.01%) spike of peanut in milk chocolate was extracted in the presence of the given amount of competitor and according to the given procedure. The extracted DNA was amplified in 8 independent calibrated real-time cqPCR reactions. The results are displayed in table 5. The 100 ppm peanut in chocolate were quantified in average at 97.8 ppm (range 81.2 to 132.0%). The quantitative variation (% CV) between 8 independent PCR reactions of one sample extraction was only 17%, which indicates a high reproducibility of the calibrated real-time cqPCR reaction. Taking into account the somewhat higher coefficient of variation between 17 and 29% on the basis of two extractions and each two amplification reactions, some degree of variation is obviously due to sample preparation.

The above described procedure of a simplified 1-point calibrated competitive quantitative real-time PCR results in a precise and accurate quantification of peanut even in different matrices and without the need of any further calibration between different matrices. Peanut was used as an example of an organic substance and chocolate as an arbitrary matrix.

The observed high reproducibility and accuracy allows to simplify the analytical set-up down to a single extraction and a single amplification reaction of an individual sample matrix in the described calibrated real-time cqPCR without substantial loss of quantitative characteristics.

### SUMMARY OF RESULTS

As a proof of concept, the normalized real-time PCR was at least as sensitive, specific and quantitative for peanut as were AOAC Research Institute Performance Tested peanut specific ELISA. Bearing in mind that peanut is the potential allergenic food for which most of the research and development of immunochemical methods has been put on, the potential power of the calibrated cqPCR method has been demonstrated. Moreover, several potentially allergenic sources may not be immunogenic enough to obtain high quality, high specificity antibodies to detect the allergenic food. By contrast, the calibrated cqPCR is independent from a successful immunization of animals and further can be designed according to oligonucleotide chemistry.

The advantage of this PCR-invention over common antibody-based techniques such as ELISA are (I) a completely reproducible chemistry including recombinant polymerases, (II) virtually infinite availability because of reproducible chemistry, (Ill) a very stable marker molecule because DNA is extraordinary heat stable in comparison to proteins, the latter of which are detected in ELISA, (IV) unparalleled specificity, (V) the avoidance of external calibration standard curves, (VI) internal calibration to normalize sample matrix effects, (VII) an internal calibrator (competitor) that also serves as inhibition control with regard to sample matrix effects, and (VIII) finally a threshold based quantification that is efficient when verifying compliance with given analytical or regulatory thresholds.

In this example, calibration of the real-time PCR device was either done with external standard curves or with a 1-point calibration step that both allow to directly compare the position of signals of target and competitor such as to i) semi-quantitatively estimate or to ii) accurately calculate the amount of organic target species in a sample matrix.

The 1-point calibration substitutes the external standard curves and allows accurate, precise and economic quantification. Moreover, the measurement of external reference samples is made unnecessary. Moreover, the quantification is made transferable and independent of the real-time thermocycler.

In this example, samples were extracted in duplicate, and each sample DNA extract was analyzed in PCR in duplicate. In total four competitive PCRs were run to obtain a quantitative result. For screening purposes to verify the presence of the targeted species, or to have a quantitative result without substantial loss of quantitative characteristics, one PCR run would suffice. Making use of modern real-time PCR thermocyclers with at least four fluorescence channels, this calibrated cqPCR further developed as a duplex analyte PCR can run in one reaction tube to quantify two different species simultaneously. Consequently, a screening of 8 different quantitative thresholds may be actualized with only 4 PCR reactions with only one additional vessel in case of applying the 1-point calibrator. In comparison to ELISAs that have external calibration curves, usually with at least 4 standard points for each analyte, the calibrated cqPCR is also economically competitive.

The calibration towards the analytical or regulatory threshold can also be achieved empirically without knowing the absolute copy number of the competitor DNA but simply by applying a serial dilution of the stock of purified generated competitor DNA. Accordingly, the difference in threshold cycle (ΔC_{T}) between competitor (HEX) and peanut (FAM) nucleic acid can be recorded at predefined fluorescence signal intensity. The ΔC_{T} is plotted against the logarithm of the factor of dilution of the serially diluted stock of the competitor DNA that has been generated as described. After linear regression of the semi-logarithmic data, the mathematical equation of the linear regression is solved for the factor of dilution of competitor DNA at ΔC_{T} equals zero. Finally, the competitor stock that is diluted according to the determined factor of dilution, serves as the internal standard to be added to any sample with unknown amount of organic substance to be quantified. Similarly, both the external standard curves and the 1-point calibration mixture of target and competitor nucleic acid may be composed empirically, based on appropriate serially diluted nucleic acid stocks of both target and competitor without knowing the true number of amplifiable copies.

A quantification of the species of interest is now made possible on the basis of nucleic acid quantification techniques, however without quantifying the nucleic acids as an unnecessary interstate.

Once the cqPCR application is calibrated and performed in a kit as described above, it enables to quantify the target organic substance in sample matrixes other than the calibrated matrix without further need of calibration for the novel matrixes. This is especially achieved by the addition of the competitor nucleic acid to the sample matrix prior to nucleic acid extraction and amplification. Moreover, the measurement of external reference samples is made unnecessary. Moreover, the quantification is made transferable and independent of the real-time thermocycler.

This cqPCR can be calibrated against any available or self-prepared reference, provided it is representative for the whole species, including the presence of the species specific DNA or RNA.

The observed high reproducibility and accuracy, even of the 1-point calibrated real-time cqPCR, allows to simplify the analytical set-up down to a single extraction and a single amplification reaction of one individual sample matrix without substantial loss of quantitative characteristics. Applying the 1-point calibration, the common 96-well format of real-time PCR thermocyclers can accommodate the simultaneous quantification of 95 different samples in different sample matrixes, making quantitative PCR economically competitive.

This format is especially powerful if integrated into a ready to use kit for the operator. This helps to reduce the steps of manipulation down to only a few steps up to full automation of the calibrated cqPCR, including sample preparation.

### Tables

TABLE 1
Quantified peanut in the spiked milk chocolate sample.

| | |
|---|---|
| spike level of peanut | verified peanut spikes |

| % | ppm (mg/kg) | ppm (mg/kg) | % CV |
|---|---|---|---|
| 10 | 100,000 | 96237 | 5.4 |
| 1 | 10,000 | 8944 | 5.9 |
| 0.1 | 1,000 | 988 | 10.0 |
| 0.01 | 100 | 75.9 | 1.8 |
| 0.001 | 10 | 6.86 | 0.5 |

(% CV, coefficient of variation of duplicate determination)

TABLE 2
Quantification of peanut spiked into milk chocolate at a level between 10 to 100,000 ppm. Identification of the quantitative working
range. (% CV, coefficient of variation; % R, recovery rate)

| | | Extraction | PCR | CT | CT | Δ CT (Hex- | | | % peanut | | |
|---|---|---|---|---|---|---|---|---|---|---|---|
| Sample | | no. | no. | (HEX) | (FAM) | Fam) | % peanut | mean t | mean | % CV | % R |
| 0.001% | peanut | 1 | 1 | 31.44 | 34.19 | −2.75 | 0.0014 | | | | |
| | | 1 | 2 | 31.62 | 34.37 | −2.75 | 0.0014 | 0.0014 | | | |
| (6.86 | ppm) | 2 | 1 | 31.38 | 35.41 | −4.03 | 0.0006 | 0.0007 | 0.00128 | 35.3 | 187 |
| | | 2 | 2 | 31.08 | 34.63 | −3.55 | 0.0008 | | | | |
| 0.01% | peanut | 1 | 1 | 31.81 | 31.84 | −0.03 | 0.0098 | | | | |
| | | 1 | 2 | 31.32 | 31.59 | −0.27 | 0.0082 | 0.0090 | | | |
| (75.9 | ppm) | 2 | 1 | 30.97 | 31.51 | −0.54 | 0.0067 | 0.0082 | 0.0086 | 4.7 | 113 |
| | | 2 | 2 | 31.20 | 31.25 | −0.05 | 0.0096 | | | | |
| 0.1% | peanut | 1 | 1 | 32.01 | 28.03 | 3.98 | 0.158 | | | | |
| | | 1 | 2 | 31.71 | 28.14 | 3.57 | 0.119 | 0.138 | | | |
| (988 | ppm) | 2 | 1 | 31.62 | 28.34 | 3.28 | 0.097 | 0.103 | 0.121 | 14.7 | 122 |
| | | 2 | 2 | 31.59 | 28.15 | 3.44 | 0.109 | | | | |
| 1% | peanut | 1 | 1 | 32.72 | 24.51 | 8.21 | 2.96 | | | | |
| | | 1 | 2 | 33.37 | 24.69 | 8.68 | 4.10 | 3.53 | | | |
| (8944 | ppm) | 2 | 1 | 32.37 | 24.22 | 8.15 | 2.84 | 3.04 | 3.29 | 7.5 | 368 |
| | | 2 | 2 | 32.61 | 24.27 | 8.34 | 3.24 | | | | |
| 10% | peanut | 1 | 1 | 36.82 | 22.16 | 14.66 | 259 | | | | |
| | | 1 | 2 | 37.76 | 22.08 | 15.68 | 525 | 392 | | | |
| (96237 | ppm) | 2 | 1 | 39.01 | 22.78 | 16.23 | 769 | 1.169 | 781 | 49.8 | >1000 |

| | | | | | |
|---|---|---|---|---|---|
| 2 | 2 | 39.58 | 22.32 | 17.26 | 1.570 |

TABLE 3
Comparative quantification of peanut by ELISA and PCR. (choc., chocolate; % R, recovery rate; * 2/4 PCR positive).

| | | ppm peanut | cqPCR | | ELISA A | | ELISA B | | ELISA C | |
|---|---|---|---|---|---|---|---|---|---|---|
| Sample type | matrix | known | ppm peanut | % R | ppm peanut | % R | ppm peanut | % R | ppm peanut | % R |
| § 64 LFGB | milk choc. | 2 | 1.62* | 81 | 1.97 | 98 | 4.2 | 211 | <LOD | |
| § 64 LFGB | Dark choc. | 20 | 17.9 | 89 | 7.2 | 36 | 13.6 | 68 | 1.1 | 5.4 |
| § 64 LFGB | milk choc. | 100 | 101 | 101 | 117 | 117 | 170.1 | 170 | 91.9 | 92 |
| Own spike | milk choc. | 6.86 | 12.8 | 187 | 8.4 | 123 | 14.3 | 208 | 1.13 | 17 |
| Own spike | milk choc. | 75.9 | 86.0 | 113 | 68.9 | 91 | 188.1 | 248 | 78.3 | 103 |
| Own spike | milk choc. | 988 | 1206 | 122 | 757 | 77 | 1884 | 191 | 908 | 92 |
| own spike | milk choc. | 0 | <LOD | | <LOD | | <LOD | | <LOD | |
| commercial | Dark choc. | unknown | <LOD | | <LOD | | <LOD | | <LOD | |
| commercial | milk choc. | unknown | <LOD | | <LOD | | <LOD | | <LOD | |
| commercial | mousse | unknown | <LOD | | <LOD | | <LOD | | <LOD | |
| commercial | milk choc. | unknown | 11.4 | | 13.4 | | 11.5 | | 0.87 | |
| commercial | nougat choc. | unknown | 47.7 | | 47.4 | | 15.2 | | 38.5 | |

TABLE 4
Matrix independency of the 1-point calibrated real-time cqPCR: Quantification of 100 ppm (0.01%) peanut in three different food
matrices on the basis of a 1-point calibration (Stdev standard deviation; CV coefficient of variation; R recovery)

| Sample | | ppm peanut | ppm peanut | | | |
|---|---|---|---|---|---|---|
| type | matrix | known | quantified | Stdev | % CV | % R |
| own spike* | milk chocolate | 100 | 97.5 | 16.7 | 17.2 | 98 |
| own spike | cookie dough | 100 | 101.8 | 27.5 | 27.1 | 102 |
| § 64 LFGB | milk chocolate | 100 | 82.9 | 24.3 | 29.4 | 83 |
| § 64 LFGB | dark chocolate | 100 | 73.6 | 17.7 | 24.0 | 74 |

*against which real-time cqPCR was calibrated

TABLE 5
Reproducibility of the 1-point calibrated real-time
cqPCR: Repetitive quantification of one DNA extraction
derived from 100 ppm (0.01%) peanut in chocolate.

| Simultaneous | | | ppm |
|---|---|---|---|
| cqPCR runs | Fluorescence | C_{T} | peanut |
| run 1 | HEX | 32.22 | |
| run 1 | FAM | 32.37 | 90.1 |
| run 2 | HEX | 32.58 | |
| run 2 | FAM | 32.49 | 106.4 |
| run 3 | HEX | 32.69 | |
| run 3 | FAM | 32.29 | 132.0 |
| run 4 | HEX | 32.42 | |
| run 4 | FAM | 32.30 | 108.7 |
| run 5 | HEX | 32.35 | |
| run 5 | FAM | 32.59 | 84.7 |
| run 6 | HEX | 32.15 | |
| run 6 | FAM | 32.35 | 87.1 |
| run 7 | HEX | 32.28 | |
| run 7 | FAM | 32.39 | 92.7 |
| run 8 | HEX | 31.87 | |
| run 8 | FAM | 32.17 | 81.2 |

1. Method for the quantitative detection of an organic substance in a sample based on a given threshold value for that organic substance in the sample through an amplification of a nucleic acid to be detected that stems from said organic substance, comprising the steps of:
a) providing a given number of a second nucleic acid as a competitor (competitor nucleic acid) in an amplification of the nucleic acid to be detected, and adding the competitor nucleic acid to the sample, wherein the given number of the competitor nucleic acid results in an amplification in a signal that allows for deducing the amount of the organic substance in the sample relative to the given threshold value,
b) extracting the nucleic acid to be detected and the competitor nucleic acid from the sample,
c) providing a first oligonucleotide and a second oligonucleotide for priming an amplification of the nucleic acid to be detected and of the competitor nucleic acid,
d) providing a first detectable probe for hybridizing with the nucleic acid to be detected to generate a first detectable signal, and a second detectable probe for hybridizing with the competitor nucleic acid to generate a second detectable signal,
e) performing an amplification of the nucleic acid to be detected and simultaneously of the competitor nucleic acid, and
f) measuring the signal from the first detectable probe and the signal from the second detectable probe and deducing therefrom the amount of the organic substance in the sample relative to the given threshold value,
wherein the amplification is performed through a real-time polymerase chain reaction.

2. Method according to claim 1, wherein the deduction of the amount of the organic substance in the sample is performed by determining the C_{T} value of the signal from the first detectable probe and of the signal from the second detectable probe representing the threshold value and comparing the C_{T} value from the first detectable probe with the C_{T} value from the second detectable probe to deduce therefrom the amount of the organic substance in the sample relative to the threshold value by application of the following steps:
in case the C_{T} value from the first detectable probe is substantially identical to the C_{T} value from the second detectable probe, it is concluded that the amount of the organic substance in the sample is identical to the threshold value,
in case the C_{T} value from the first detectable probe is below the C_{T} value from the second detectable probe, it is concluded that the amount of the organic substance in the sample is higher than the threshold value,
in case the C_{T} value from the first detectable probe is above the C_{T} value from the second detectable probe, it is concluded that the amount of the organic substance in the sample is lower than the threshold value.

3. Method according to claim 2, wherein if PCR efficiency for both the organic substance and the competitor is similar or identical and nearly 100%, a difference in C_{T} value from the first detectable probe to the C_{T} value from the second detectable probe by 3.322 cycles results in a relative difference of tenfold compared to the calibrated amount.

4. Method according to claim 1, wherein the nucleic acid to be detected and the competitor nucleic acid both comprise a first sequence portion for hybridization with the first oligonucleotide, and a second sequence portion for hybridization with the second oligonucleotide, wherein the first sequence portion of the nucleic acid to be detected or of the competitor nucleic acid contains a sequence that is reverse complementary to the sequence of the first oligonucleotide, or the second sequence portion of the nucleic acid to be detected or of the competitor nucleic acid contains a sequence that is reverse complementary to the sequence of the second oligonucleotide.

5. Method according to claim 4, wherein the first sequence portion of the nucleic acid to be detected and the first sequence portion of the competitor nucleic acid are identical; or
wherein the second sequence portion of the nucleic acid to be detected and the second sequence portion of the competitor nucleic acid are identical; or
wherein the amplification product of the nucleic acid to be detected and of the competitor nucleic acid are identical in length; or
wherein the amplification product of the nucleic acid to be detected and the competitor nucleic acid differ in sequence in at least 1 nucleotide; or
wherein the nucleic acid to be detected and the competitor nucleic acid differ in sequence only at a sequence portion where the detectable probe hybridizes.

6. Method according to claim 1, wherein the first detectable probe and the second detectable probe are tagged with a detectable label, wherein the detectable label of the first detectable probe is distinguishable from the detectable label of the second detectable probe.

7. Method according to claim 1, wherein the quantitative detection is an absolute quantitative detection.

8. Packaging unit or kit for the quantitative detection of an organic substance in a sample wherein a target nucleic acid, which presence correlates with the amount of organic substance, is isolated from said sample and quantified by real-time PCR to determine whether the organic substance was comprised above or below a set threshold value in said sample, comprising:
at least one vessel for calibration of the detection system comprising defined amounts of said target nucleic acid and a competitor nucleic acid in such a ratio that their C_{T}-values can be laid over each other when quantified in a standardised real-time PCR;
at least one vessel comprising a defined amount of competitor nucleic acid to be added to a defined amount of sample to be extracted, or at least one extraction vessel comprising a defined amount of competitor into which a defined addition of the sample to the defined amount of competitor nucleic acid is done prior to nucleic acid extraction, so that the defined amount of competitor nucleic acid serves as an internal standard for nucleic acid extraction and amplification from said sample and is chosen such that the amount of competitor sets an internal reference to the quantitative threshold of the organic substance when a defined amount of sample is analysed by nucleic acid extraction and real-time PCR with respect to the presence of target and competitor nucleic acids; and
optionally, one or more amplification vessels comprising first and second primers, reagents and enzymes for nucleic acid extraction and for performing a standardized real-time PCR.

9. Packaging unit or kit according to claim 8, further comprising one or more vessels for holding said sample and subsequent nucleic acid extraction, each vessel comprising a defined amount of competitor nucleic acid that sets an internal threshold when the target and competitor nucleic acids are analysed by real-time PCR.

10. Packaging unit or kit according to claim 8, wherein the amplification vessels further comprise first and second detectable probes which hybridise with the target nucleic acid and the competitor nucleic acid respectively, and which are respectively labelled with first and second detectable labels chosen such that the first detectable label of the first probe is distinguishable from the second detectable label of the second probe.

11. Packaging unit or kit according to claim 8, wherein the defined amount of competitor nucleic acid is incorporated i) in a liquid solution phase, or ii) as a material reversibly bound to an enclosed solid phase to be added or iii) in a glassy layer of trehalose bound to the vessel walls.

12. Packaging unit or kit according to claim 8, wherein the defined amounts of competitor nucleic acid and target nucleic acid are incorporated i) in a liquid solution phase, or ii) as a material reversibly bound to an enclosed solid phase to be added or iii) in a glassy layer of trehalose bound to the vessel walls of the calibration vessels.

13. Packaging unit or kit according to claim 8, wherein the reaction vessels are appropriately sized micro-reaction tubes or wells of a microtiter plate.

14. Packaging unit or kit for the simultaneous quantitative detection of two or more organic substances in a sample wherein respective target nucleic acids, the presence of which correlates with the amount of organic substances, are isolated from said sample and quantified by real-time PCR to determine whether the organic substances were comprised at, above, or below a set threshold value in said sample, comprising:
at least one vessel for calibration of the detection system comprising defined amounts of said target nucleic acids and respective competitor nucleic acids in such a ratio that their respective C_{T}-values can be laid over each other when quantified in a standardised real-time PCR;
at least one vessel comprising defined amounts of competitor nucleic acids to be added to a defined amount of sample to be extracted, or at least one extraction vessel comprising defined amounts of competitors into which a defined addition of the sample to the defined amounts of competitor nucleic acids is done prior to nucleic acid extraction, so that the defined amounts of competitor nucleic acids serve as an internal standards for nucleic acid extraction and amplification from said sample and are chosen such that the amounts of competitor set internal references to the quantitative thresholds of the organic substances when a defined amount of sample is analysed by nucleic acid extraction and real-time PCR with respect to the presence of target and competitor nucleic acids; and
optionally, one or more vessels comprising primers, probes, reagents and enzymes for nucleic acid extraction and for performing a standardized real-time PCR;
wherein the probes are respectively labelled with detectable labels comprising probes chosen such that all the detectable labels are distinguishable, so as to allow relative quantification of each target nucleic acid compared to its respective competitor nucleic acid.

15. (canceled)

16. Method according to claim 5, wherein the amplification product of the nucleic acid to be detected and the competitor nucleic acid differ in sequence in 1 to 20 nucleotides.

17. Packaging unit or kit according to claim 10, wherein the first and second detectable labels are chosen from the group consisting of FAM, HEX, Cy3 and Cy5.
